(19) United States
(12) Patent Application Publication
(10) Pub. No.: US 2024/0173711 A1
(43) Pub. Date: May 30, 2024

(54) CATALYTIC REACTION PROCESS

(71) Applicant: UOP LLC, Des Plaines, IL (US)

(72) Inventors: John Q. Chen, Morton Grove, IL (US); Wei Pan, Hoffman Estates, IL (US); Stephen C. Houdek, Des Plaines, IL (US)

(21) Appl. No.: 18/483,573

(22) Filed: Oct. 10, 2023

Related U.S. Application Data
(60) Provisional application No. 63/385,587, filed on Nov. 30, 2022.

(51) Int. Cl. B01J 38/16; B01J 21/04; B01J 23/42; B01J 23/96; B01J 38/02; C07C 5/333

(52) U.S. Cl. CPC B01J 38/16; B01J 21/04; B01J 23/42; B01J 23/96; B01J 38/02; C07C 5/3337; C07C 2523/04; C07C 2523/08; C07C 2523/42

(57) ABSTRACT

A system for contacting a reactant stream with a catalyst that includes a reactor containing a quantity of catalyst and an inlet and a product outlet configured for discharging product and a catalyst regenerator unit having an inlet configured for receiving a spent catalyst stream from the reactor and an outlet configured for passage of a regenerated catalyst to the reactor where a water level in the reactor is about 10 vppm to about 3 mole percent.

### RELATED APPLICATIONS

This application claims priority to U.S. Provisional Patent Application Ser. No. 63/385,587 filed on Nov. 30, 2022, the entirety of which is incorporated herein by reference.

### FIELD OF THE INVENTION

This invention relates generally to a catalytic reaction system, and more particularly, to an improved catalytic reaction process.

### BACKGROUND OF THE INVENTION

Catalysis is the process of increasing the rate of a chemical reaction by adding a substance known as a catalyst. Catalysts generally react with one or more reactants in a reactor to form intermediate products that subsequently result in a final reaction product.

Light olefin production is a process that utilizes a catalyst and is vital to the production of sufficient plastics to meet worldwide demand. Paraffin dehydrogenation (PDH) is a process in which light paraffins such as ethane, propane and butanes can be dehydrogenated to make ethylene, propylene and butenes, respectively, where the reaction is an endothermic reaction that requires external heat to drive the reaction. In dehydrogenation reactions and similar reactions, achieving higher catalyst activity and selectivity during the reaction improves the resulting products.

Certain dehydrogenation catalysts are negatively impacted by the presence of water in the process stream during a reaction. Lowering the concentration of water, i.e., the mole percentage of water in the gas phase, in the reaction process results in a significant increase in catalyst activity. However, completely removing water from the reaction process, negatively impacts catalyst selectivity. Lower catalyst selectivity leads to higher cost of production due to loss of feedstock to undesirable by-products and increases the cost of product separation.

Accordingly, there is a need for controlling the water level in a catalytic reaction process to improve catalyst reactivity and catalyst selectivity and the resulting products.

### SUMMARY OF THE INVENTION

The present system and process is directed to a catalytic reaction process including a reactor and/or a catalyst regeneration unit that adjusts a water level in the system and process to improve catalyst reactivity and selectivity in the reaction process to enhance the resulting products from the reaction system and process.

Thus, the present invention may be broadly characterized as providing a system for contacting a reactant stream with a catalyst where the system comprises a reactor containing a quantity of catalyst and an inlet and a product outlet configured for discharging product and a catalyst regenerator unit having an inlet configured for receiving a spent catalyst stream from the reactor and an outlet configured for passage of a regenerated catalyst to the reactor, where a water level in the reactor gas phase is about 10 vppm to about 3 mole percent. In an embodiment, a water level in the reactor is about 100 vppm to about 2 mole percent. In another embodiment, a water level inside the reactor is about 200 vppm to about 1 mole percent. In an embodiment, the reactor includes an inlet configured for receiving a reactant, the reactant including a water level of about 10 vppm to about 3 mole percent. In another embodiment, a temperature of the catalyst regenerator unit is 550° C. to 900° C. such that a water level of the regenerated catalyst is about 100 wppm to about 8000 wppm. In a further embodiment, the system further comprises at least one water level sensor at the inlet of the reactor or inside the reactor, where the water level sensor is in communication with a control unit. In an embodiment, the system further comprises at least one water level sensor at the product outlet of the reactor, where the water level sensor being in communication with a control unit. In an embodiment, the system further comprises at least one water level sensor at the outlet of the catalyst regenerator unit, where the water level sensor being in communication with a control unit. In another embodiment, the system further comprises at least one water level sensor at the inlet of the reactor, inside the reactor, at the product outlet of the reactor and at the outlet of the catalyst regenerator unit, each water level sensor being in communication with a control unit. In an embodiment, the catalyst comprises an active metal dispersed on a porous inorganic carrier material, the porous inorganic carrier being one of silica, alumina, silica alumina, zirconia, clay or zeolite. In a further embodiment, the catalyst comprises at least one of alumina, silica-alumina, a noble metal, an alkali, an alkaline earth metal and gallium.

A second embodiment of the invention is a process comprising receiving a reactant stream in a reactor, contacting the reactant stream with a catalyst in the reactor, separating spent catalyst from the product stream, outputting a product stream from the reactor, regenerating spent catalyst in a catalyst regenerator unit, wherein the spent catalyst is received from the reactor, supplying regenerated catalyst to the reactor from the catalyst regenerator unit and controlling a water level in the reactor to enhance catalyst activity and selectivity, wherein the water level is about 10 vppm to about 3 mole percent. In an embodiment, the water level in the reactor is about 100 vppm to about 2 mole percent. In a further embodiment, the water level in the reactor is about 200 vppm into about 1 mole percent. In an embodiment, a water level of the reactant stream is about 10 vppm to about 3 mole percent. In another embodiment, the process further comprises heating the catalyst regenerator unit to a temperature of 550° C. to 900° C. such that a water level of the regenerated catalyst is about 100 wppm to about 8000 wppm. In an embodiment, the process further comprises sensing a water level sensor at an inlet of the reactor, inside the reactor, at an outlet of the reactor and at an outlet of the catalyst regenerator unit. In an embodiment, the catalyst comprises an active metal dispersed on a porous inorganic carrier material, the porous inorganic carrier being one of silica, alumina, silica alumina, zirconia, clay or zeolite. In another embodiment, the catalyst comprises at least one of alumina, silica-alumina, a noble metal, an alkali, an alkaline earth metal and gallium.

Additional aspects, embodiments, and details of the invention, all of which may be combinable in any manner, are set forth in the following detailed description of the invention.

### DETAILED DESCRIPTION OF THE DRAWINGS

One or more exemplary embodiments of the present invention will be described below in conjunction with the following drawing figures, in which:

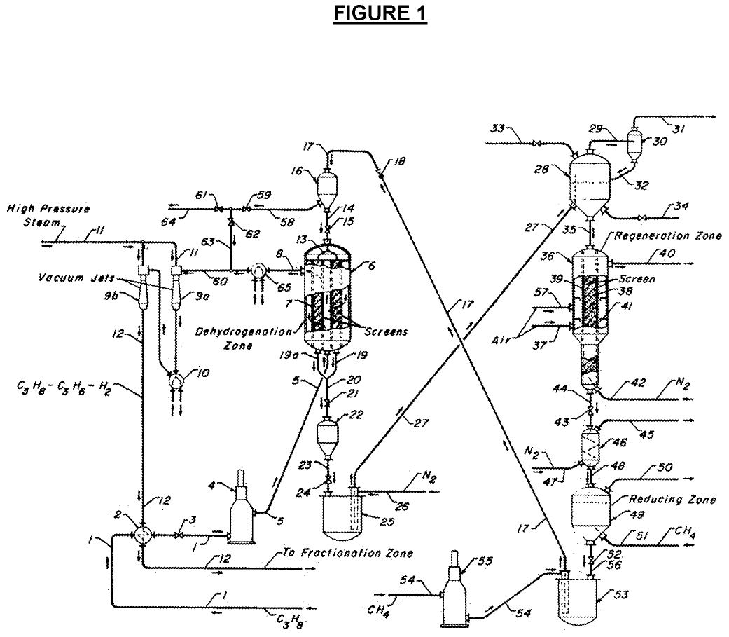

FIG. 1 is a perspective view of a catalyst reactor system;

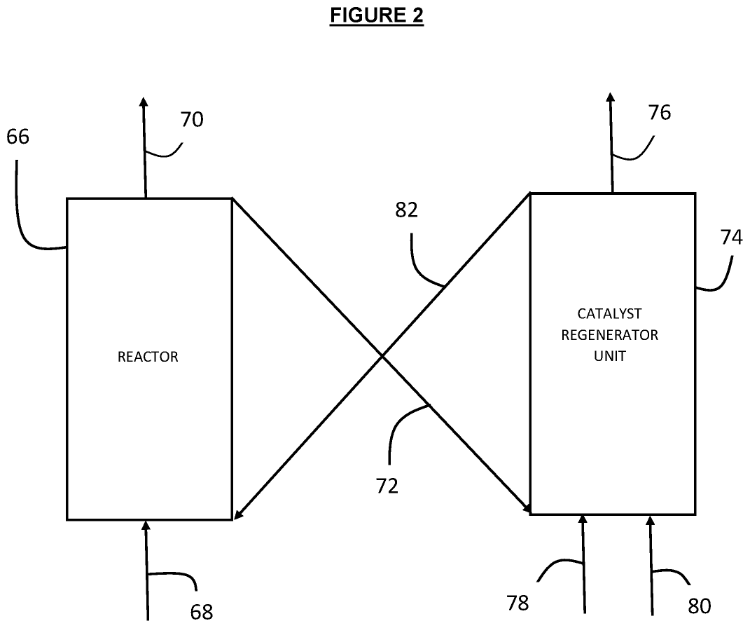

FIG. 2 is a schematic view of a reactor and a catalyst regenerator unit;

### DETAILED DESCRIPTION OF THE INVENTION

Referring to FIG. 1, an embodiment of a reactor system that utilizes a catalyst to produce a product is shown. The reactor uses the catalyst to contact a reactant entering the reactor as a feed stream to cause a reaction within the reactor and produce a resulting product. The reaction within the reactor may be any suitable chemical reaction that uses a catalyst.

In an embodiment, the operation of the reactor system will be described in terms of the dehydrogenation of propane, but this is not intended to limit the scope of the invention. A feed stream comprising propane enters the process through line 1 and is heated in feed-effluent heat exchanger 2. The feed stream then passes through a flow control valve 3 and enters a furnace 4. The feed stream is heated to the desired inlet temperature and passed through line 5 into a dehydrogenation reactor 6. The feed stream enters the central portion of the reaction zone, which is maintained at a sub atmospheric pressure, and passes outwardly through an annular bed of catalyst 7. The contacting of the heated feed stream with the catalyst effects the dehydrogenation of a portion of the feed stream and the production of olefins and hydrogen. The olefins, hydrogen and unconverted propane exit the reaction zone through line 8 as the effluent stream of the process. Heat is recovered from the effluent stream in heat exchanger 65. The effluent stream is admixed with a smaller stream of methane passing at times through line 63 and is then passed into line 60. The effluent stream then enters the first of two vacuum jets 9a and 9b which are driven by high pressure steam entering through line 11. Interstage cooling is provided through heat exchange means 10. The effluent stream then passes through line 12 and the feed-effluent heat exchanger 2 to a fractionation zone for the recovery of the products. Unconverted propane may be recycled through line 1.

The useful life of the dehydrogenation catalyst is relatively short, and the catalyst within the reaction zone is gradually replaced with fresh catalyst and/or regenerated catalyst, to allow continuous operation of the process. Small discrete quantities of catalyst are withdrawn through a multitude of catalyst withdrawal lines represented by lines 19 and 19a and passed into a central collection line 20. The rate of catalyst withdrawal is controlled by valve means 21. The withdrawn catalyst falls into a collection hopper 22 wherein the catalyst is pressurized by a means not shown. The accumulated catalyst is then passed from the lock hopper 22 to a lift engager 25 through line 23 by opening valve means 24. Valve means 24 is once again closed and a gas stream comprising nitrogen or flue gas is passed through line 26 to cause the transfer of the catalyst upward through line 27.

Spent catalyst is withdrawn from the reaction zone in the reactor and is sent to a catalyst regenerator unit having a collection hopper 28, where the catalyst is separated from the gas stream. The separated gases exit the top of the collection hopper through line 29 and enter into a cyclone separator 30. This results in a further separation of catalyst from the gases and produces an off-gas stream comprising fine catalyst particles which is removed in line 31. The larger reusable catalyst particles are returned to the collection hopper through line 32. Fresh catalyst is added through line 33 to make up for breakage, and catalyst is removed through line 34 when a replacement or change of catalyst is desired.

Catalyst is withdrawn from the collection hopper 28 by gravity-flow through line 35. The catalyst then enters a regeneration zone 36 wherein it is retained as a dense cylindrical bed of catalyst 38 located within a screen 39. A first stream of air enters the regeneration zone through line 37 and is forced to pass upward through the catalyst bed 38 by the baffle means 41. A second stream of air enters through line 57 and also passes upward through the catalyst bed. Contacting of the hot catalyst with the air stream results in the burning off of the carbon layers on the catalyst. Excess air and the combustion products separate from the catalyst and are withdrawn through line 40. A small continuous stream of nitrogen enters the bottom of the regeneration zone through line 42 and is passed upward through the lower part of catalyst bed 38 to strip air from the catalyst.

In the illustrated embodiment, catalyst is withdrawn either intermittently or continuously through line 44 at a rate controlled by valve means 43. The catalyst then falls into a stripping vessel 46 wherein it is contacted by a second nitrogen stream entering through line 47. This nitrogen stream and the stripped gases are removed through line 45. The catalyst then falls through line 48 into a reducing zone 49. A small continuous stream of methane is fed into the bottom of the reducing zone through line 51. This results in the reduction of the metals contained on the catalyst. The gaseous effluent of the reducing zone is removed through line 50. The at least partially reduced catalyst is then removed from the reducing zone through line 56 at a rate controlled by valve 52 and passed into a second lift engager 53. A stream of methane entering the process through line 54 is passed through a second furnace 55, which heats it to a temperature substantially equal to that of the reaction zone. This gas stream is then passed into the lift engager to cause the transfer of the catalyst upward through line 17 and to ensure its total reduction. The catalyst passes through a valve means 18 and enters an upper lock hopper 16. The heated methane passes downward through the catalyst and is removed via lines 58 and 64 through open valves 59 and 61. Valves 18 and 61 are then closed, and valve 62 in line 63 is opened to equalize the pressures within the lock hopper and within the reactor 6. Valve means 15 is then opened to allow the transfer of catalyst through lines 14 and 13 at a rate which is controlled by its withdrawal through lines 19a and 19.

The above description is directed to a dehydrogenation reaction of propane. It should be appreciated that the above process may be used for paraffin dehydrogenation reactions, a paraffin dehydrocyclization reaction, a naphthalene dehydrogenation reaction or any type of dehydrogenation or dehydrocyclization reaction.

Light olefin production is vital to the production of sufficient plastics to meet worldwide demand. Paraffin dehydrogenation (PDH) is a process in which light paraffins such as ethane, propane, and butane can be dehydrogenated to make ethylene, propylene, and butenes respectively. Dehydrogenation is an endothermic reaction which requires external heat to drive the reaction to completion.

In PDH reactions using a fluidized catalyst, coke can deposit on the catalyst while catalyzing the reaction, and the dehydrogenation metals may be deactivated by extended contact with reaction conditions. The catalyst may be regenerated in a catalyst regenerator by combusting coke from the catalyst in the presence of oxygen. The hot regenerated catalyst may then be transferred back to the reactor to catalyze the reaction. If insufficient heat is provided to drive the endothermic reaction, olefin production can suffer. For Pt-Ga catalyst, to maintain the catalyst activity, regeneration of catalyst is accomplished by burning supplemental fuel and then subjugating the carbon-burned catalyst to additional oxygen treatment in each cycle to maintain catalyst activity over time. Catalyst stripping for water removal may be part of the catalyst regenerator.

Referring to FIG. 2, an embodiment of a PDH reaction process is shown where the process includes a PDH reactor 66 and a feed line 68 that delivers a reactant stream to an inlet of the reactor 66. The reactant stream may predominantly comprise propane or butane, but other paraffins such as ethane may be present in the reactant stream in conjunction with or to the exclusion of other paraffins. Any feed distributor can distribute the reactant stream to the reactor 66. A product stream 70, such as a mixture of propylene and hydrogen, exits an outlet of the reactor 66. A spent catalyst stream 72 exits the reactor 66 and is sent to a bottom inlet to the catalyst regenerator unit 74. In connection with catalyst regenerator unit 74, a regeneration flue gas 76 exits out a top outlet of the catalyst regenerator unit 74. A regeneration gas stream 78 and a fuel stream 80, enter a bottom inlet or inlets to the catalyst regenerator unit 74. A regenerated catalyst stream 82 is returned to the reactor 66 from an outlet of the catalyst regenerator unit 74. The catalyst regenerator unit 74 may optionally include an oxygen treatment zone for catalyst conditioning or rejuvenation and/or a stripping zone for water and oxygen removal from the catalyst.

The present process enhances catalyst reactivity and selectivity by adjusting water levels in the reaction process, and more specifically, in the reactor. In an embodiment, the conditions in the dehydrogenation reactor include a temperature of about 500° C. to about 800° C., a pressure of about 40 kPa to about 310 kPa and a catalyst to feed ratio of about 5 to about 100. The dehydrogenation reaction is conducted in a fluidized manner such that gas, which may comprise the reactant paraffins with or without a fluidizing inert gas, is distributed to the reactor in a way that lifts the dehydrogenation catalyst in the reactor vessel while catalyzing the dehydrogenation of paraffins. During the catalytic dehydrogenation reaction, coke is deposited on the dehydrogenation catalyst leading to reduction of the activity of the catalyst. The dehydrogenation catalyst must then be regenerated and returned to the reactor as described above.

The dehydrogenation catalyst may be any type of catalyst suitable for a fluidized bed dehydrogenation unit. The dehydrogenation catalyst selected should minimize cracking reactions and favor dehydrogenation reactions. Suitable catalysts for use herein include an active metal which may be dispersed in on a porous inorganic carrier material such as silica, alumina, silica alumina, zirconia, clay or zeolite. An exemplary embodiment of a catalyst includes alumina or silica-alumina containing gallium, a noble metal, and an alkali or alkaline earth metal.

In the above process, a catalyst support comprises a carrier material, a binder and an optional filler material to provide physical strength and integrity to the catalyst. The carrier material may include alumina or silica-alumina. Silica sol or alumina sol or other sources may be used as the binder precursor. The alumina or silica-alumina generally contains alumina of gamma, theta and/or delta phases. The catalyst support particles may have a nominal diameter of about 20 to about 200 micrometers with the average diameter of about 50 to about 150 micrometers. Preferably, the surface area of the catalyst support is 85 to 170 m²/g.

The dehydrogenation catalyst may contain a dehydrogenation metal. The dehydrogenation metal may be a transition metal or a combination of transition metals. In an embodiment, the dehydrogenation catalyst is a noble metal, such as platinum or palladium. In another embodiment, the catalyst is gallium, which is an effective metal for paraffin dehydrogenation. The catalyst metal or metals may be deposited on the catalyst support by impregnation or other suitable methods or included in the carrier material or binder during catalyst preparation.

The acid function of the catalyst is minimized to prevent cracking and favor dehydrogenation. Alkali metals and alkaline earth metals may also be included in the catalyst to attenuate the acidity of the catalyst. Also, rare earth metals may be included in the catalyst to control the activity of the catalyst. Concentrations of 0.001 weight percent or wt % to 10 wt % metals may be incorporated into the catalyst. In the case of the noble metals, it is preferred to use about 1 parts per million (ppm) by weight to about 600 ppm by weight of a noble metal. More preferably it is preferred to use 2 ppm to 100 ppm by weight of a noble metal. In an embodiment, the preferred noble metal is platinum. Further, gallium is present in the range of about 0.3 wt % to about 10 wt %, and preferably about 1 wt % to about 7 wt %. Alkali and alkaline earth metals are also present in the range of about 0.05 wt % to about 1 wt %.

As described above, the stripped, spent dehydrogenation catalyst is transported by a spent catalyst pipe to the catalyst regenerator unit to combust the coke on the spent catalyst and regenerate the spent catalyst into regenerated catalyst. An oxygen supply gas is provided which lifts the spent catalyst in a chamber through a catalyst separator and into a separation chamber. The coke is burned off the spent catalyst by contact with the oxygen supply gas at regeneration conditions. In an exemplary embodiment, air is used as the oxygen supply gas, because air is readily available and provides sufficient oxygen for combustion. Exemplary regeneration conditions include a temperature from about 550° C. to about 900° C., preferably from about 670° C. to about 750° C., and a pressure of about 103 kPa (abs) (15 psia) to about 450 kPa (abs) (70 psia) in the catalyst regenerator unit. Hydrocarbon fuel may be added to the catalyst regenerator unit to boost the heat generated in the catalyst regenerator unit and helps to provide heat in the reactor.

The above embodiments provide a catalytic reaction process and apparatus for contacting a reactant stream with a fluidized catalyst and further provides catalyst regeneration and rejuvenation to maintain catalyst activity and catalyst selectivity.

### EXAMPLE 1

A catalyst was prepared by spray drying. The spray dried catalyst contains 100 weight ppm of Pt, 1.7 weight percent of Ga, 0.30 weight percent of K, 1.1 weight percent of SiO₂, and remaining balance as Al₂O₃. The catalyst is pre-aged for 315 cycles in a PDH-Regeneration multi-cycle aging test.

Long-term aging of catalyst was simulated by cycling the catalyst between reactor and regenerator conditions as follows:

Startup: 1 cm³ of catalyst was loaded in a quartz tube reactor. Catalyst was heated to 120° C. in nitrogen and held 30 minutes. Temperature was increased to 720° C. in nitrogen at 10° C./min and regeneration conditions were initiated.

Regeneration step: Temperature was increased to 720° C. in nitrogen at 10° C./min. Gas composition was changed from nitrogen to 5% O₂, 24.2% H₂O, balance N₂ (by volume) and was flowed for 2 minutes with gas flow rate of 15 standard cubic cm per minute. Gas composition was changed back to nitrogen, temperature was held for 0.5 min, and cooling was initiated.

Reaction step: Sample was cooled to 620° C. in nitrogen at 13° C./min. Gas composition was changed from nitrogen to propane. Propane was flowed for 2 minutes with gas flow rate of 15 cm³ per minute. Gas composition was changed back to nitrogen, temperature was held for 0.5 min, and heating for regeneration step was initiated.

A total of 315 cycles were completed, with an additional regeneration at the end of the program. Catalyst was cooled in nitrogen and unloaded for further testing.

### EXAMPLE 2

In another example, 52 mg of the catalyst from Example 1, which was cycle-aged for 315 cycles, was pre-mixed with 1100 mg of spray dried inert alpha alumina diluent loaded between quartz wool plugs in a quartz tube reactor with inner diameter 4.1 mm. Inert alpha alumina spheres were loaded below the catalyst bed to minimize thermal cracking. The reactor effluent composition was analyzed by transmission infrared spectroscopy MKS analyzer which identified propane, propene, ethane, ethene, and methane products. The effluent of the infrared analyzer was directed to a gas chromatograph which was used to occasionally analyze the product stream and check the accuracy of the infrared analyzer on the real product stream.

Each experiment consists of 5 reaction/regeneration cycles with N₂ purge step between reaction step and regeneration step. Cycles 1-2 has no H₂S pre-adsorbed on the catalyst while Cycles 3-5 has H₂S pre-adsorbed on the catalyst as detailed below.

Step (a) Regeneration Heating/Combustion Step:

The catalyst is treated at 720° C. for 1.5 minutes with a gaseous mixture with molar composition of 5% O₂/8% CO₂/20% H₂O/67% N₂. This gaseous mixture simulates composition of a gas composition within a PDH regenerator after combustion of a fuel source and at least a portion of coke contained on the catalyst. Total flow rate of the gaseous mixture is equivalent to 27 ml/min as measured at room temperature and pressure.

Step (b) Stripping Step:

Substitute the gas in Step (a) with a dry N₂ gas flow at 20 ml/min and reactor temperature is lowered to 620° C. Once reactor temperature reaches 620° C., switch to a N₂ gas stream that contains the same level of H₂O as in PDH cycle and hold at the temperature for additional 60 minutes. Water is introduced by passing N₂ gas through a water saturator.

For cycles 1 and 2, PDH step is initiated right after the 60 minutes N₂ stripping. For cycles 3, 4 and 5, an additional step of H₂S pre-adsorption is added after the 60 minutes N₂ stripping. This is achieved by adding 3.8 ml/min of (50 vppm H₂S/N₂) gas to the N₂ stripping stream (combined total flow of 23.8 ml/min with 8 vppm H₂S) for 10 minutes. The H₂S/N₂ stream is turned off after 10 minutes and the catalyst is purged with N₂ for another 10 minutes before initiating PDH step.

Step (c) PDH Step:

Substitute the gas in Step b) with 100% propane feed at propane weight hourly space velocity (WHSV) of 35.1 reciprocal hour (hr-1). Effect of H₂O level, Time on Stream (TOS) and H₂S pre-adsorption are studied as detailed below. Water is introduced by passing propane feed through a H₂O saturator. MKS total hydrocarbon signal intensity vs time is recorded. Time zero is determined by extrapolating initial linear part of the MKS total hydrocarbon intensity vs time response curve to MKS signal intensity of 0.

Step (d) Purge Step:

Substitute the gas in Step (c) with a dry N₂ flow at 20 ml/min. Raise reactor temperature to 720° C. Repeat Step (a) for the next cycle test.

TABLE 1
Experimental Details

| | Reactor | H₂O partial | | |
|---|---|---|---|---|
| | outlet | pressure in | H₂O | |
| Experiment | pressure | propane feed | concentration | Pre-adsorbed |
| # | (psi) | (psi) | (mole %) | H₂S |
| 1 | 20.7 | 0.00 | 0 | Cycles 1-5 w/o |
| | | | | H₂S |
| 2 | 21.0 | 0.15 | 0.63 | Cycles 1-2 w/o |
| | | | | H₂S |
| | | | | Cycles 3-5 |
| | | | | w/H₂S |
| 3 | 20.7 | 0.30 | 1.26 | Cycles 1-2 w/o |
| | | | | H₂S |
| | | | | Cycles 3-5 |
| | | | | w/H₂S |

Conversion is calculated by following equation:

$$\frac{\begin{matrix}( Propane in feed , carbon mole ) - \\ ( Propane in product , carbon mole )\end{matrix}}{( Propane in feed , carbon mole )} \times 100 \%$$

Propylene selectivity is calculated by following equation:

$$\frac{( Propylene , carbon mole )}{( All hydrocarbon reaction products , carbon mole )} \times 100 \%$$

Table 2 below compares the catalytic conversion of the catalyst with feed streams containing moisture partial pressures of 0 psi, 0.15 psi, and 0.30 psi, where the catalytic conversion (catalyst contribution in the absence of any thermal cracking) is calculated by subtracting thermal contributions measured separately under identical process conditions from observed overall conversion. Table 3 below compares the catalytic selectivity at similar catalytic conversion of propane, where the catalytic selectivity is calculated by removing thermal cracking contributions.

TABLE 2
Catalytic conversion with feed containing different moisture levels

| | Reactor | H₂O partial | | Catalytic conversion (%) at | | | | |
|---|---|---|---|---|---|---|---|---|
| | outlet | pressure in | H₂O | 0.55 minute-on-stream | | | | |
| Experiment | pressure | propane feed | concentration | 1^{st} | 2^{nd} | 3^{rd} | 4^{th} | 5^{th} |
| # | (psi) | (psi) | (mole %) | cycle | cycle | cycle | cycle | cycle |
| 1 | 20.7 | 0.00 | 0.0 | 38.7 | 39.0 | 39.5 | 41.6 | 40.5 |
| 2 | 21.0 | 0.15 | 0.63 | 21.8 | 21.0 | 20.2 | 17.5 | 19.4 |
| 3 | 20.7 | 0.30 | 1.26 | 12.2 | 11.8 | 12.8 | 12.2 | 12.8 |

TABLE 3
Catalytic selectivity at 31% catalytic conversion of propane
with feeds containing different moisture levels

| | Reactor | H₂O partial | | Catalytic selectivity at 31% | | | | |
|---|---|---|---|---|---|---|---|---|
| | outlet | pressure in | H₂O | catalytic conversion | | | | |
| Experiment | pressure | propane feed | concentration | 1^{st} | 2^{nd} | 3^{rd} | 4^{th} | 5^{th} |
| # | (psi) | (psi) | (mole %) | cycle | cycle | cycle | cycle | cycle |
| 1 | 20.7 | 0.00 | 0.0 | 98.5 | 98.6 | 98.5 | 98.6 | 98.5 |
| 2 | 21.0 | 0.15 | 0.63 | 100 | 100 | 100 | 100 | 100 |
| 3 | 20.7 | 0.30 | 1.26 | 100 | 100 | 100 | 100 | 100 |

In the above embodiments, a control system including a controller or a control unit, which may be a processor, is used to control the operation of the reaction process, and more specifically, the reactor furnace and/or the catalyst regenerator unit, such that the control unit communicates with the reactor furnace and/or the catalyst regenerator unit and is designed to actively control the injection rates, locations, localized stoichiometry of the reaction process.

In an embodiment, the control system (controller or control unit) includes a control panel located in a control room at the facility including the reaction process or at a remote site that is at a different facility or location from the reaction process, and communicates with the reactor furnace and/or the catalyst regenerator unit with a Distributed Control System (DCS) via Modbus TCP/IP (Transmission Control Protocol/Internet Protocol) communications. During operation, the control system provides orderly and safe startup, operation, and shutdown of the reaction process.

Any of the above lines, conduits, units, devices, vessels, surrounding environments, zones or similar may be equipped with one or more monitoring components including sensors, measurement devices, data capture devices or data transmission devices. Signals, process or status measurements, and data from monitoring components may be used to monitor conditions in, around, and on process equipment. Signals, measurements, and/or data generated or recorded by monitoring components may be collected, processed, and/or transmitted through one or more networks or connections that may be private or public, general or specific, direct or indirect, wired or wireless, encrypted or not encrypted, and/or combination(s) thereof; the specification is not intended to be limiting in this respect.

Signals, measurements, and/or data generated or recorded by monitoring components may be transmitted to one or more computing devices or systems. Computing devices or systems may include at least one processor and memory storing computer-readable instructions that, when executed by the at least one processor, cause the one or more computing devices to perform a process that may include one or more steps. For example, the one or more computing devices may be configured to receive, from one or more monitoring component, data related to at least one piece of equipment associated with the process. The one or more computing devices or systems may be configured to analyze the data. Based on analyzing the data, the one or more computing devices or systems may be configured to determine one or more recommended adjustments to one or more parameters of one or more processes described herein. The one or more computing devices or systems may be configured to transmit encrypted or unencrypted data that includes the one or more recommended adjustments to the one or more parameters of the one or more processes described herein.

The computing device of system unit may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory may be any suitable known or other machine-readable storage medium. The memory may comprise non-transitory computer readable storage medium such as, for example, but not limited to, an electronic, magnetic, optical, electromagnetic, infrared, or semiconductor system, apparatus, or device, or any suitable combination of the foregoing. The memory may include a suitable combination of any type of computer memory that is located either internally or externally to the device such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. The memory may comprise any storage means (e.g., devices) suitable for retrievably storing the computer-executable instructions executable by the controller or a computing device.

The methods and steps described herein may be implemented in a high-level procedural or object-oriented programming or scripting language, or a combination thereof, to communicate with or assist in the operation of the controller or computing device. Alternatively, the methods and systems described herein may be implemented in assembly or machine language. The language may be a compiled or interpreted language. Program code for implementing the methods and systems for control gas flow to a burner described herein may be stored on the storage media or the device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein.

Computer-executable instructions may be in many forms, including program modules, executed by one or more computers or other devices. Generally, program modules include routines, programs, objects, components, data structures, etcetera, that perform particular tasks or implement particular abstract data types. Typically, the functionality of the program modules may be combined or distributed as desired in various embodiments.

### SPECIFIC EMBODIMENTS

While the following is described in conjunction with specific embodiments, it will be understood that this description is intended to illustrate and not limit the scope of the preceding description and the appended claims.

A first embodiment of the invention is a system for contacting a reactant stream with a catalyst where the system comprises a reactor containing a quantity of catalyst and an inlet and a product outlet configured for discharging product and a catalyst regenerator unit having an inlet configured for receiving a spent catalyst stream from the reactor and an outlet configured for passage of a regenerated catalyst to the reactor, where a water level in the reactor is about 10 vppm to about 3 mole percent. An embodiment of the invention in one, any or all of prior embodiments in this paragraph up through the first embodiment in this paragraph, where a water level in the reactor is about 100 vppm to about 2 mole percent. An embodiment of the invention in one, any or all of prior embodiments in this paragraph up through the first embodiment in this paragraph, where a water level in the reactor is about 200 vppm to about 1 mole percent. An embodiment of the invention in one, any or all of prior embodiments in this paragraph up through the first embodiment in this paragraph, where the reactor includes an inlet configured for receiving a reactant, the reactant including a water level of about 10 vppm to about 3 mole percent. An embodiment of the invention in one, any or all of prior embodiments in this paragraph up through the first embodiment in this paragraph, where a temperature of the catalyst regenerator unit is 550° C. to 900° C. such that a water level of the regenerated catalyst is about 100 wppm to about 8000 wppm. An embodiment of the invention in one, any or all of prior embodiments in this paragraph up through the first embodiment in this paragraph, further comprising at least one water level sensor at the inlet of the reactor or inside the reactor, where the water level sensor is in communication with a control unit. An embodiment of the invention in one, any or all of prior embodiments in this paragraph up through the first embodiment in this paragraph, further comprising at least one water level sensor at the product outlet of the reactor, where the water level sensor being in communication with a control unit. An embodiment of the invention in one, any or all of prior embodiments in this paragraph up through the first embodiment in this paragraph, further comprising at least one water level sensor at the outlet of the catalyst regenerator unit, where the water level sensor being in communication with a control unit. An embodiment of the invention in one, any or all of prior embodiments in this paragraph up through the first embodiment in this paragraph, further comprising at least one water level sensor at the inlet of the reactor, inside the reactor, at the product outlet of the reactor and at the outlet of the catalyst regenerator unit, each water level sensor being in communication with a control unit. An embodiment of the invention in one, any or all of prior embodiments in this paragraph up through the first embodiment in this paragraph, where the catalyst comprises an active metal dispersed on a porous inorganic carrier material, the porous inorganic carrier being one of silica, alumina, silica alumina, zirconia, clay or zeolite. An embodiment of the invention in one, any or all of prior embodiments in this paragraph up through the first embodiment in this paragraph, where the catalyst comprises at least one of alumina, silica-alumina, a noble metal, an alkali, an alkaline earth metal and gallium.

A second embodiment of the invention is a process comprising receiving a reactant stream in a reactor, contacting the reactant stream with a catalyst in the reactor, separating spent catalyst from the product stream, outputting a product stream from the reactor, regenerating spent catalyst in a catalyst regenerator unit, wherein the spent catalyst is received from the reactor, supplying regenerated catalyst to the reactor from the catalyst regenerator unit and controlling a water level in the reactor to enhance catalyst activity and selectivity, wherein the water level is about 10 vppm to about 3 mole percent. An embodiment of the invention in one, any or all of prior embodiments in this paragraph up through the second embodiment in this paragraph, where the water level in the reactor is about 100 vppm to about 2 mole percent. An embodiment of the invention in one, any or all of prior embodiments in this paragraph up through the second embodiment in this paragraph, where the water level in the reactor is about 200 vppm to about 1 mole percent. An embodiment of the invention in one, any or all of prior embodiments in this paragraph up through the second embodiment in this paragraph, where a water level of the reactant stream is about 10 vppm to about 3 mole percent. An embodiment of the invention in one, any or all of prior embodiments in this paragraph up through the second embodiment in this paragraph, further comprising heating the catalyst regenerator unit to a temperature of 550° C. to 900° C. such that a water level of the regenerated catalyst is about 100 wppm to about 8000 wppm. An embodiment of the invention in one, any or all of prior embodiments in this paragraph up through the second embodiment in this paragraph, further comprising sensing a water level sensor at an inlet of the reactor, inside the reactor, at an outlet of the reactor and an outlet of the catalyst regenerator unit. An embodiment of the invention in one, any or all of prior embodiments in this paragraph up through the second embodiment in this paragraph, where the catalyst comprises an active metal dispersed on a porous inorganic carrier material, the porous inorganic carrier being one of silica, alumina, silica alumina, zirconia, clay or zeolite. An embodiment of the invention in one, any or all of prior embodiments in this paragraph up through the second embodiment in this paragraph, where the catalyst comprises at least one of alumina, silica-alumina, a noble metal, an alkali, an alkaline earth metal and gallium.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A system for contacting a reactant stream with a catalyst, the system comprising:
a reactor containing a quantity of catalyst and an inlet and a product outlet configured for discharging product; and
a catalyst regenerator unit having an inlet configured for receiving a spent catalyst stream from said reactor and an outlet configured for passage of a regenerated catalyst to said reactor,
wherein a water level in said reactor is about 10 vppm to about 3 mole percent.

2. The system of claim 1, wherein a water level in said reactor is about 100 vppm to about 2 mole percent.

3. The system of claim 1, wherein a water level in said reactor is about 200 vppm to about 1 mole percent.

4. The system of claim 1, wherein said reactor includes an inlet configured for receiving a reactant, said reactant including a water level of about 10 vppm to about 3 mole percent.

5. The system of claim 1, wherein a temperature of said catalyst regenerator unit is 550° C. to 900° C. such that a water level of said regenerated catalyst is about 100 wppm to about 8000 wppm.

6. The system of claim 1, further comprising at least one water level sensor at said inlet of said reactor or inside said reactor, said water level sensor being in communication with a control unit.

7. The system of claim 1, further comprising at least one water level sensor at said product outlet of said reactor, said water level sensor being in communication with a control unit.

8. The system of claim 1, further comprising at least one water level sensor at said outlet of said catalyst regenerator unit, said water level sensor being in communication with a control unit.

9. The system of claim 1, further comprising at least one water level sensor at said inlet of said reactor, inside said reactor, at said product outlet of said reactor and at said outlet of said catalyst regenerator unit, each said water level sensor being in communication with a control unit.

10. The system of claim 1, wherein the catalyst comprises an active metal dispersed in a porous inorganic carrier material, said porous inorganic carrier being one of silica, alumina, silica alumina, zirconia, clay or zeolite.

11. The system of claim 1, wherein the catalyst comprises at least one of alumina, silica-alumina, a noble metal, an alkali, an alkaline earth metal and gallium.

12. A process comprising:
receiving a reactant stream in a reactor;
contacting the reactant stream with a catalyst in said reactor;
outputting a product from said reactor;
regenerating spent catalyst in a catalyst regenerator unit, wherein said spent catalyst is received from said reactor;
supplying regenerated catalyst to said reactor from said catalyst regenerator unit; and
controlling a water level in said reactor to enhance catalyst activity and selectivity, wherein the water level is 10 vppm to 3 mole percent.

13. The process of claim 12, wherein the water level in said reactor is about 100 vppm to about 2 mole percent.

14. The process of claim 12, wherein the water level in said reactor is about 200 vppm into about 1 mole percent.

15. The process of claim 12, wherein a water level of the reactant stream is about 10 vppm to about 3 mole percent.

16. The process of claim 12, further comprising heating said catalyst regenerator unit to a temperature of 550° C. to 900° C. such that a water level of said regenerated catalyst is about 100 wppm to about 8000 wppm.

17. The process of claim 12, further comprising sensing a water level sensor at an inlet of said reactor, inside said reactor, at an outlet of said reactor and an outlet of said catalyst regenerator unit.

18. The process of claim 12, wherein the catalyst comprises an active metal dispersed in a porous inorganic carrier material, said porous inorganic carrier being one of silica, alumina, silica alumina, zirconia, clay or zeolite.

19. The process of claim 12, wherein the catalyst comprises at least one of alumina, silica-alumina, a noble metal, an alkali, an alkaline earth metal and gallium.
